(19) United States
(12) Patent Application Publication
(10) Pub. No.: US 2012/0183626 A1
(43) Pub. Date: Jul. 19, 2012

(54) ANTISEPTIC OINTMENT COMPRISING BENTONITE INTERCALATED WITH SILVER, COPPER OR ZINC FOR EXTERNAL APPLICATION

(76) Inventors: Vladimir Ilych Barbakov, Moscow (RU); Viacheslav Ivanovich Beklemyshev, Moscow (RU); Evgeniya Fedorovna Beklemysheva, Moscow (RU); Igor Ivanovich Makhonin, Mendeleevo (RU); Umberto Orazio Giuseppe Maugeri, Calvignano (IT); Irina Mihailovna Meshkova, Mendeleevo (RU); Vladimir Aleksandrovich Solodovnikov, Moscow (RU)

(21) Appl. No.: 13/261,228

(22) PCT Filed: Aug. 17, 2010
(86) PCT No.: PCT/EP2010/061992

§ 371 Date: Mar. 23, 2012

(30) Foreign Application Priority Data
Sep. 24, 2009 (RU) 2009135436

(51) Int. Cl. A61K 33/38; A61K 33/30; A61P 31/00; A61K 33/34

(52) U.S. Cl. 424/618; 424/630; 424/642

(57) ABSTRACT

The invention concerns antiseptic ointments for external application comprising, as a basis, pharmaceutical Vaseline or a mix of pharmaceutical Vaseline with lanoline, and, as the antiseptic agent, a dispersed bentonite powder intercalated by one or more metal ions selected in the group consisting of Ag⁺, Cu²⁺, Zn²⁺. In addition the ointment contains an etheric vegetable oil. More precisely the active antiseptic agent may be: a dispersed bentonite powder intercalated by ions of Ag⁺ or by ions of Cu²⁺ or by a mix of said bentonites. According to an anternative the antiseptic dispersed bentonite powder may be intercalated by ions of Ag⁺ and Zn²⁺ or by ions of Cu²⁺ and Zn²⁺ or by ions of Ag⁺, Cu⁺ and Zn⁺. In any case the total content of metals in the composition of ointment is comprised between 0.05-0.5%. The ointments according to the invention are biologically compatible with various integuments of humans and animals and provide a prolonged antiseptic action at low expenses.

### FIELD OF THE INVENTION

The invention concerns the chemical-pharmaceutical industry and can be applied in medicine and veterinary science for manufacturing of antiseptic ointments for external application, possessing antimicrobic and antifunguses action and intended for treatment of diseases of integuments of various aetiology in humans and animals.

### PRIOR ART

At present time various ointments including ointments with antiseptic agents on the basis of copper, silver and zinc-containing components, providing a wide range of antimicrobic actions, are applied for treatment of affections of integuments of various aetiology in humans and animals.

In medical practice and veterinary science the antimicrobic action of the named metals and also of Au, Pt, Pd is widely known (see H. E. Morton, Pseudomonas in Disinfection, Sterilisation and Preservation, ed. S. S. Block, Lea and Febider 1977 and N. Grier, Silver and Its Compounds in Disinfection, Sterilisation and Preservation, ed. S. S. Block, Lea and Febiger, 1977).

In particular, the ointments containing silver sulfadiazine and piperacillin sodium (U.S. Pat. No. 4,535,078 of 1985) or wounds healing materials on the basis of the animal tissues impregnated with silver salt of sulfadiazine (U.S. Pat. No. 4,599,226 of 1987) are known.

Common shortcomings of the given medical products, including habit-forming of microorganisms, are the following:
presence of contra-indications because of the collateral effects peculiar to sulpha drugs: leukopenia, allergies, dyspeptic disorders;
inexpediency of their use for treatment of purulent wounds and burned surfaces with plentiful exudation.

In the technical specification of patent RU N. 2146127, an ointment containing the carrier as gel of polyethyleneoxide, a bactericidal component as atomic colloidal silver and the stabilizer of the last component as polyvinylpyrrolidone and polyethyleneoxide, is described for more rapid healing of infected wounds.

However application of colloidal silver in the form of ointment demands injection of additional components in its structure as stabilizers of ointment system to avoid agglomeration of silver particles. That increases expenses for manufacturing ointment.

In the specification of patent RU N. 2331407 a gel is described in liposomal form of water-soluble substances containing an antiseptic agent on the basis of electrolytic silver, obtained in a electrolyzer filled with electrolyte as a liquid phase of the sodium-acetate buffer and flat silver electrodes with a content of silver at 99.99%.

Application of energy is an expensive way of obtaining electrolytic silver and also the technological processes for obtaining a gel in liposomal forms of water-soluble substances, as described in the above named Russian patent, increases expenses for manufacturing of the compositions according to the invention.

Ointment with antiseptic properties, containing a carrier as polyethyleneglycol and a mix of salts of copper acetate Cu(CH₃COO)₂, H₂O and silver acetate (AgC₂H₃O₂), at a ratio 1:0,1, is described for treatment and preventive maintenance of otituses of alveary in the patent RU N. 2297840. The mix of salts is a donor of ions of silver and copper in the generated composition of ointment.

Though the specified compounds are effective as soluble salts, they do not provide long protection due to a decrease in antiseptic action owing to complex-formation of free ions of silver and copper.

Application of a zinc-containing component in antiseptic ointments for external application (see Patent RU N. 2160089) is also well known. According to it, ointment contains the carrier as a fatty basis, active components as vitamin A, D and E, salicylic acid, zinc oxide, etc. Application of ointment is intended for decreasing inflammatory processes of integuments.

The given antiseptic ointment contains a significant part of zinc oxide. It has a number of components which increase expenses for its manufacturing.

It also follows from known documents (see patent RU 2131269 of 10.06.1999 -WO 94046003/14), that the solution of the technical problem of increasing activation of Ag, Cu and Zn ions in a water dispersion of metal powders, can be realized at presence of significant linear and point defects in a crystal lattice of said metals. These defects provide irregularity of surface and structural heterogeneities at nanometer level. However processes for obtaining such structures as nanocrystal powder are based on cold machining (grinding, crushing, mealing) at temperatures lower than their recrystallization temperature, what requires development of special technologic modalities, causing increase of expenses.

Thus, from the state of the art, as a whole it follows that known antiseptic ointments for external application, with use of antiseptic agents on the basis of silver, copper and zinc-containing components do not correspond to the requirement of obtaining highly effective antiseptic means with prolonged action, at low level of expenses for their manufacturing.

At the same time a highly effective antiseptic agent, created on the basis of inorganic components is known (see patent RU N. 2330673). The antiseptic agent in the form of a dispersed bentonite powder intercalated by ions of Ag⁺ or/and by ions of Cu²⁺ is described in the above named patent. The ions are obtained by modifying a bentonite with solutions of inorganic salts such as silver nitrate or copper sulfate. Bentonite is preliminarily enriched by cations of Na⁺ by processing with a water solution of inorganic salts of sodium and subsequent cleaning from anions.

From the above patent it follows that the antiseptic agent consisting of a bentonite powder intercalated by ions of Ag⁺ or/and by ions of Cu2⁺ is obtained from mineral, ecologically safe components. They are biologically compatible with tissues of living organisms and can be applied as additives in various sectors including medicine and veterinary science for antimicrobic processing of the injured zones of integuments, for example, in composition with various ointment bases or gels.

However in the given patent the composition of ointment for external application, including for processing of hard healing wounds, trophic ulcers, burns, dermatosis and pustular diseases of a skin, is not resolved.

Besides, the given antiseptic agent on the basis of a mix of bentonite powders intercalated by ions of Ag⁺ and by ions of Cu²⁺ in a structure of ointment for external application can provide an allergy at processing of integuments of patients with diabetes.

An antiseptic agent for elimination of the given shortcomings is described in the Russian patent application No. 2009117737, with priority of 13.05.09, of the same Applicants, as follows:
mixes of dispersed bentonite powders intercalated by ions Ag⁺ and Zn²⁺ at a ratio 1:(0.2-0.8) or
mixes of bentonite powders intercalated by ions Ag⁺, Zn²⁺ and Cu²⁺ at a ratio 1:(0.2-0.8):(0.2-0.5) or
mixes of bentonite powders intercalated by ions of Zn²⁺ and Cu²⁺ at a ratio 1:(0.2-0.5).
   The mixes are obtained by modification of bentonite with water solutions of silver nitrate, copper sulfate, zinc chloride or zinc sulfate. Bentonite is preliminarily enriched with cations of Na⁺ by processing with a water solution of a inorganic salt of sodium and subsequent cleaning from anions.

Presence of the antiseptic agent as a dispersed bentonite powder intercalated by ions of zinc in the given composition promotes inhibition of allergenic processes at processing of integuments.

However the compounding of antiseptic ointment for external application is not concretized also in the given technical specification.

The technical specification of patent RU No. 2330673 is chosen as the closest prior art for antiseptic ointment according to the first variant and the technical specification of the application RU No. 2009117737 is chosen as the closest prior art for ointments according to the second variant.

### SUMMARY OF THE INVENTION

Choosing the nearest prior art documents of the present invention, the inventors considered the following elements:
the composition of ointment contains at least two basic components in the antiseptic ointments according to the prior art, namely: the basis-carrier and the antiseptic agent, which functional features provide technical properties of the ointment at its external application;
the basis-carrier with the given antiseptic agent is applied in the preparation of the compositions on the basis of disperse bentonite powders intercalated by ions of the above mentioned metals. It promotes effective distribution of the composition on work surfaces and reduces expenses for manufacturing of the composition with preservation of effective antiseptic activity.

The purpose of the given inventions was the creation of ointments for external application providing the technical result of prolonged antiseptic action, at low expenses, of active components present in its structure. Thus, active components are biologically compatible with various integuments of humans and also animals.

### DETAILED DESCRIPTION OF THE INVENTION

Antiseptic ointment for external application according to the first variant, with the basis and the antiseptic agent as a disperse bentonite powder intercalated by ions of Ag⁺ or/and by ions of Cu²⁺ is described for the solution of the declared technical problem. The ions are obtained by modification of bentonite with water solutions of silver nitrate or copper sulfate. Bentonite is preliminarily enriched with cations of Na⁺ by processing with a water solution of a inorganic salt of sodium and subsequent cleaning from anions. In accordance with inventions medical vaseline or a mix of medical vaseline with lanolin is used as a basis in a ratio of parts by weight as 1:(0.1-0.5). In addition ointment contains etheric vegetable oil and has the following content of components (weight %):

| | |
|---|---|
| bentonite powder intercalated by ions of Ag⁺ or | 1-10 |
| bentonite powder intercalated by ions of Cu²⁺ or | |
| a mix of mentioned powders at a ratio 1:(0.1-0.4) | |
| oil | 0.01-0.3 |
| basis | the rest, |

thus the total content of metals in the composition of ointment is 0.05-0.5 weight %.

Antiseptic ointment for external application, according to the second variant of the invention, with the basis and the antiseptic agent as a mix of disperse bentonite powders intercalated by ions of Ag⁺ and Zn²⁺ at a ratio 1:(0.2-0.8), or a mix of bentonite powders intercalated by ions of Ag⁺, Zn²⁺ and Cu²⁺ at a ratio 1:(0.2-0.8):(0.2-0.5), or a mix of bentonite powders intercalated by ions of Zn²⁺ and Cu²⁺ at a ratio 1:(0.2-0.5) is described as a solution of the declared technical problem. The ions are obtained through modification of bentonite by water solutions of silver nitrate, copper sulfate, zinc sulfate or zinc chloride. Bentonite is preliminarily enriched with cations of Na⁺ by processing with a water solution of a inorganic salt of sodium and subsequent cleaning from anions.

In accordance with the invention, medical vaseline or a mix of medical vaseline with lanolin is applied as a basis at a ratio 1:(0.1-0.5).

In addition the ointment contains etheric vegetable oil and has the following content of components (weight %):

| | |
|---|---|
| a mix of bentonite powders | 1-10 |
| oil | 0.01-0.3. |
| basis | the rest. |

Thus the total content of metals in the composition of ointment is 0.05-0.5 weight %.

Above mentioned inorganic salts of silver, copper and zinc, at a ratio of bentonite:solution as 1:(10-40) are used for intercalation of bentonite enriched by ions of Na⁺ in accordance with the invention.

According to the invention bentonite powders intercalated by ions of silver, copper, zinc have a weight fraction of the specified metals of 2-6 weight %

In accordance with the invention pink oil or oil of sage, or their mix at a ratio 1:(0.1-0.5) is used as etheric vegetable oil.

At realization of the invention
due to the presence of etheric vegetable oil in the composition of ointment, antiseptic, anesthetizing and anti-inflammatory properties of ointment are improved at treatment of skin diseases;
due to the presence of antiseptic agents as dispersed bentonite powders intercalated by ions of silver, copper and zinc, efficiency of their distribution in the named fatty basis of ointment is provided. Bigger area of contact of the mentioned agents with the bacterial environment raises efficiency of antimicrobic and antifunguses influences on pathogenic microflora;
due to presence of the basis as medical vaseline or a mix of medical vaseline with lanolin, softening and protective action on integuments is provided.

When used in a composition according to the present invention the ratio of the components corresponds to the requirements of their synergetic compatibility.

It was not found any patent describing the set of characteristics corresponding to the present declared technical solution and realizing the above described result of prolonging action of bactericidal (antimicrobic) and antifunguses efficiency on integuments of warm-blooded organisms, at the analysis of technical experts.

The result of analysis of the technical experts testifies the conformity of the declared technical solution to criteria of “novelty” and “degree of inventiveness”.

The declared invention can be industrially realized for obtaining antiseptic ointments intended, for example, for treatment of wounded, burned, ulcered zones of integuments, of various aetiology.

The components ready to application are used for realization of the invention:
etheric vegetable oils; oil pink as the most effective on the anti-inflammatory properties is more preferable; medical vaseline (GOST 3582-84 or FS 42-2456-97); waterless lanolin;
a bentonite powder intercalated by ions of silver, with mass fractions of silver not less than 2%-TR 1794-001-99699175-2007;
bentonite powders intercalated by ions of Cu²⁺ and Zn²⁺ obtained according to known patents (see patent RU No. 2330673 and patent application RU No. 2009117737—Patent holder—CSC “Institute of Applied Nanotechnology”).
   The equipment, processes and raw materials as specified in the given patent documents are used for manufacturing the mentioned bentonite powders and among them:
bentonite (montmorillonite) in Na-form, for example Sarigyuh deposit (Armenia); it is an alkaline bentonite with contents of montmorillonite (bentonite in Na-form) about 75-85% b.w. At choosing the given mineral, the inventors verified that all clay minerals possess certain capacity of cationic exchange. This value is the important characteristic of a mineral and it designates the quantity of exchange cations capable to be replaced by cations of other type referred to 100 g of clay. Montmorillonite possesses the highest capacity of cationic exchange among clay minerals (up to 150 mg eqv/100 g.);
15-20% water solutions of silver nitrate (AgNO₃), copper sulfate (CuSO₄), zinc chloride (ZnCl₂) (the most accessible salts); sodium chloride (NaCl); deionized water.

The process of intercalation of bentonite enriched with ions of sodium, preliminarily cleaned from anions (washing in deionized water, filtering, drying) with the specified solutions of salts was carried out at a ratio bentonite:solution as 1:(20-40).

Powders of bentonite, obtained after intercalation with ions of silver, copper, zinc, were cleaned from salts of sodium, dried and ground.

Dispersiveness of particles of bentonite powders obtained according to the given patents intercalated by ions of the specified metals, was determined with application of electronic microscope. The size of particles of bentonite powders, after the process of their intercalation, cleaning in deionized water from salts of sodium, drying and dispersion in water, was basically no more than 150 nanometers.

Mass fraction of silver, copper and zinc, in intercalated by ions of metals bentonite powders, was determined with application of titirimetric analysis.

In particular a mix of sulfuric and nitric acids was used as reagent of decomposition of analized samples, when carrying out the titirimetric analysis for defining the % b.w. of the content of silver in the bentonite powder (TR 1794-001-99699175-2007); a solution of ammonium thiocyanate (or potassium) was used as a solution-titrant; a solution of ferric alum was used as the indicator.

It was established, as a result of the carried out researches, that the bentonite powders obtained according to the known patents contain from 2 up to 6% b.w. of silver, copper and zinc, depending on the properties of the initial raw product, quantity of activating and modifying reagents.

The specified % b.w. content of the mentioned metals in bentonite powders is optimum. The increase in the % b.w. content of them leads to increase of expenses while, a decrease in the % b.w. content of the mentioned metals in the intercalated bentonite powder, antiseptic activity of products is decreased.

At realization of the present application, an ointment for external application with the components forming an inexpensive synergetic composition, is provided. Application of the new composition provides highly effective bactericidal prolonging action, with anti-allergic effect on integuments of tissues of warm-blooded individuals, including treatment of the various infected wounds which do not react to treatment with known means.

Ointment for external application according to the invention is not toxic, does not cause allergy, has no contra-indications and possesses high antiedematous, sorption, ion-exchanging and anti-inflammatory properties.

Realization of the invention changing the composition of the applied components and their set weight ratio, will lead to deterioration of therapeutic properties of ointment or to increase in expenses for the process of its manufacturing.

Experimental Part

Realization of the invention is explained in detail by the following concrete examples, where various recipes are given for the preparation of 50 g of ointment.

### EXAMPLE 1

| | | |
|---|---|---|
| a bentonite powder intercalated with ions of Ag⁺, | 2.5 | g |
| with a content of silver in the powder of 4% b.w. | | |
| pink oil | 0.075 | ml, |
| medical vaseline the rest up to | 50 | g of ointment. |
| The content of silver in the ointment | | |
| was 0.2% b.w. | | |

### EXAMPLE 2

| | | |
|---|---|---|
| mix of bentonite powders intercalated | 2.5 | g., |
| by ions of Ag⁺ and Cu²⁺ in a ratio 1:0.2. | | |
| The content of silver in the bentonite | | |
| powder was 4% b.w., the content of | | |
| copper was 3.5% b.w., | | |
| pink oil | 0.075 | ml, |
| mix of medical vaseline and waterless | 50 | g. of ointment. |
| lanoline, in a ratio 1:0.3, the rest to | | |
| The total content of metals in the ointment | | |
| was −0.375% b.w. | | |

### EXAMPLE 3

| | | |
|---|---|---|
| mix of bentonite powders intercalated by | 2.55 | g |
| ions of Ag⁺ and Zn²⁺, in a ratio 1:0.5 | | |
| The total content of silver in the bentonite | | |
| powder was −2% b.w.; the content of | | |
| zince was −5% b.w., | | |
| pink oil | 0.075 | ml, |
| medical vaseline - the rest up to | 50 | g. of ointment. |
| The total content of metals in the | | |
| ointment was 0.355% b.w. | | |

### EXAMPLE 4

| | | |
|---|---|---|
| mix of bentonite powders intercalated by | 3.4 | g |
| ions Ag⁺, Zn²⁺, and Cu²⁺ in the ratio 1:0, 5:0.2 | | |
| The total content of silver in the bentonite | | |
| powder was 2%, b.w.; the content of zinc 2% | | |
| b.w.; the content of copper 2% b.w. | | |
| pink oil | 0.075 | ml, |
| medical vaseline - the rest to | 50 | g. of ointment. |
| The total content of metals in the ointment | | |
| was 0.2% b.w.. | | |

### EXAMPLE 5

| | | |
|---|---|---|
| a bentonite powder intercalated by ions of Cu²⁺ | 2.0 | g. |
| The total content of copper - 2% b.w., | | |
| pink oil | 0.05 | ml, |
| medical vaseline - the rest to | 50 | g. of ointment. |
| The total content of copper was 0.08% b.w.. | | |

For manufacturing the antiseptic ointments according to the examples 1-5, the components specified in the given examples were mixed among them, at a temperature not lower than 20° C., up to formation of a homogeneous paste. Prepared ointment was packaged in vessels with a volume of no more than 50 g with strictly closed covers.

Ointment possesses high stability and high quality. It is characterized by full biocompatibility. It is preferable to keep the ointment in a dry place, protected from light, at a temperature not lower than +5° C.

Antiseptic properties of ointments made according to examples 1-5, were estimated from their activity on the most typical pathogenic microorganisms:
gram-positive and gram-negative bacteria, accordingly: Staphylococcus aureus;

Pseudomonas auruginosa;
culture of funguses: Aspergillus niger.

Microorganisms were cultivated on beef-extract broth up to a concentration of 10-9 cfu/ml. Then inoculation of cultures in firm nutrient medium of Saburo was carried out for cultivation of Aspergillus niger. Inoculation in a beef-extract agar was carried out for other cultures.

Standard disks of 5 mm diameter, treated with the samples of examples 1-5 to be tested, were imposed on the surface of a nutrient medium preliminarily inoculated with one of test-microorganisms.

The physiological solution was used as the control, by adding it in quantity of 0.05 ml on a nutrient medium. Incubation of cultures took 24-36 hours for bacteria and 5-7 days for micellar funguses.

Antiseptic activity of ointments was determined by the presence of a zone of growth inhibition of test-microorganisms in mm (by measurement of diameter) around of the above mentioned disks, in a place of their inoculation. Researches have shown high activity of ointments in respect of all applied cultures of bacteria and funguses.

Diameters of the zones of growth inhibition of test-microorganisms (Staphylococcus aureus; Pseudomonas auruginosa) in mm around of the standard disks treated with the ointments of examples 1-5, were 12 mm, according to an average analysis. The greatest diameter of the zones of growth inhibition of test-microorganisms was determined when using ointments of examples 1-3.

The data obtained from the control example have shown the presence of growth of the specified test-microorganisms in the zone of plating of the physiological solution.

Growth of test-microorganisms (Aspergillus niger), when using the compositions of examples 1-5 was not revealed within 6 days.

The determination of antiseptic properties of ointments for external application according to the invention was also estimated by means of their application on patients:
Example 1.1 A patient whose diagnosis was acne. There were complaints of the patient for plural inflamed elements on the skin.
Treatment: It was prescribed to apply the ointment of the example 1 daily for ten days.
Result: after 7 days the inflamed elements decreased of 10 times; the redness and hypostasis disappeared.
Example 1.2. A teenager 12 years old. The anamnesis: there was a wart of 3 mm diameter on the left palm.
Treatment: the wart was painted with application of the ointment of the example 2 daily for one month.
Result: the wart turned black after one month. Then the wart was painted for two days; on the third day of the second month the wart fell off. The place where there was the wart was covered with a new thin skin.
Example 1.3. A patient, whose diagnosis is diabetes with additional purulent process of soft tissues of a shin. The analysis of contents of the wound has shown the presence of gram-positive and gram-negative microflora.
Treatment:
   application of bandages covered with a layer of the ointment of example 4;
   daily change of bandages with preliminary processing of the wound by UV-radiation for 3-10 minutes with removal of suppurations and nonviable tissues.
Result: at the third day the wound was cleaned. Inoculation is sterile. After 5 days active epithelization appeared.

The effect after application of antiseptic ointment according to the invention is explained by application of the dispersed bentonite powder in the declared form of ointment.

The bentonite powder is a biologically active sorbent of purulent-necrotic masses and other products of disintegration of tissues. At interaction with various products of disintegration of tissues simultaneously, reactions of ionic exchange with cations of the metals located in the layered structure of intercalated bentonite take place, what provides antiseptic prolonged influence on the treated integuments affected by various microorganisms.

1. Antiseptic ointment for external application comprising a basis and dispersed thereon as active antiseptic agent a bentonite powder intercalated by at least one metal ion, selected in the group consisting of Ag⁺, Cu²⁺, Zn²⁺, the total content of metals in the ointment being comprised between 0.05 and 0.5% by weight.

2. Antiseptic ointment according to claim 1, wherein the basis consists of pharmaceutical Vaseline alone or in admixture with lanoline.

3. Antiseptic ointment according to claim 1, wherein the active antiseptic agent is a dispersed bentonite powder intercalated by ions of Ag⁺.

4. Antiseptic ointment according to claim 1, wherein the active antiseptic agent is a dispersed bentonite powder intercalated by ions of Cu²⁺.

5. Antiseptic ointment according to claim 1, wherein the active antiseptic agent is a dispersed bentonite powder intercalated by ions of Ag⁺ and Cu²⁺.

6. Antiseptic ointment according to claim 1, wherein the active antiseptic agent is a dispersed bentonite powder intercalated by ions of Ag⁺ and Zn²⁺.

7. Antiseptic ointment according to claim 1, wherein the active antiseptic agent is a dispersed bentonite powder intercalated by ions of Zn²⁺and Cu²⁺.

8. Antiseptic ointment according to claim 1, wherein the active antiseptic agent is a dispersed bentonite powder intercalated by ions of Ag⁺, Zn²⁺ and Cu²⁺.

9. Antiseptic ointment according to claim 5, wherein the ratio of bentonite powder intercalated by ions of Ag⁺ to the bentonite powder intercalated by ions of Cu²⁺ is of 1:(0.1-0.4).

10. Antiseptic ointment according to claim 6, wherein the ratio of bentonite powder intercalated by ions of Ag⁺ to the bentonite powder intercalated by ions of Zn²⁺ is of 1:(0.2-0.8).

11. Antiseptic ointment according to claim 7, wherein the ratio of bentonite powder intercalated by ions of Zn²⁺ to the bentonite powder intercalated by ions of Cu²⁺ is of 1:(0.2-0.5).

12. Antiseptic ointment according to claim 8, wherein the ratio among the bentonite powders intercalated by ions of Ag⁺, by ions of the Zn²⁺ and by ions of Cu²⁺ is of 1:(0.2-0.8):(0.2-0.5).

13. Antiseptic ointment according to claim 2, wherein the basis consists of a mixture of pharmaceutical vaseline and lanoline in a ratio by weight of 1:(0.1-0.5).

14. Antiseptic ointment according to claim 1, wherein the content of the bentonite powders on the total if the ointment is of 1-10 parts by weight.

15. Antiseptic ointment according to claim 14, wherein the content of the metals Ag, Cu, Zn, in the form of intercalated bentonite powders, is of 2-6% by weight on the total of ointment.

16. Antiseptic ointment according to claim 1 further comprising an etheric vegetable oil in an amount on the total of 0.01-0.5% by weight.

17. Antiseptic ointment according to claim 15, wherein the etheric vegetable oil is pink oil or sage oil or a mix thereof in a ration of 1:(0.1-0.5).
